(19) United States
(12) Patent Application Publication
(10) Pub. No.: US 2002/0193331 A1
(43) Pub. Date: Dec. 19, 2002

(54) Non-naturally occurring nucleic acid compositions, their use for the preparation of formulations useful for transfecting a nucleic acid into cells and applications

(76) Inventors: Otmane Boussif, Strasbourg (FR); Pierre Vierling, Falicon (FR); Catherine Santaella, Saint Maime (FR); Jerome Gaucheron, Orgeres en Beauce (FR)

Correspondence Address: BURNS DOANE SWECKER & MATHIS L L P, POST OFFICE BOX 1404, ALEXANDRIA, VA, 22313-1404, US

(21) Appl. No.: 09/958,142

(22) PCT Filed: Jan. 25, 2001
(86) PCT No.: PCT/EP01/00837

(30) Foreign Application Priority Data
Feb. 7, 2000 (EP) 00440034.7
Mar. 3, 2000 (US) 60186743

(51) Int. Cl. A61K 48/00

(52) U.S. Cl. 514/44

(57) ABSTRACT

Described is a composition comprising:

(i) a nucleic acid of interest; and

(iii) a special compound, or a combination of compounds, the formula of which is specifically disclosed.

Furthermore, the invention relates to the use of such a composition for transferring nucleic acids into cells.

The present invention relates to a non-naturally occurring nucleic acid composition and its use for the transfection of a nucleic acid into a cell. Such a composition is useful in gene therapy, including gene vaccination, and any therapeutic or prophylactic situation in which a gene-based product is administered to such a cell in vitro, ex vivo or in vivo.

Gene therapy has generally been conceived as principally applicable to heritable deficiency diseases (cystic fibrosis, dystrophies, haemophilias, etc.) where permanent cure may be effected by introducing a functional gene. However, a much larger group of diseases, notably acquired diseases (cancer, AIDS, multiple sclerosis, etc.) might be treatable by transiently engineering host cells to produce beneficial proteins.

Applications are, for example, the treatment of muscular dystrophies or of cystic fibrosis. The genes of Duchenne/Becker muscular dystrophy and cystic fibrosis have been identified and encode polypeptides termed dystrophin and cystic fibrosis transmembrane conductance regulator (CFTR), respectively. Direct expression of these genes within, respectively, the muscle or lung cells of patients should contribute to a significant amelioration of the symptoms by expression of the functional polypeptide in targeted tissues. Moreover, in cystic fibrosis studies have suggested that one would need to achieve expression of the CFTR gene product in only about 5% of lung epithelial cells in order to significantly improve the pulmonary symptoms.

Another application of gene therapy is vaccination. In this regard, the immunogenic product encoded by the nucleic acid introduced in cells of a vertebrate may be expressed and secreted or be presented by said cells in the context of the major histocompatibility antigens, thereby eliciting an immune response against the expressed immune polynucleotide.

Success of gene therapy depends on the efficient delivery of the nucleic acid of interest into cells of a subject to be treated. This delivery process generally means that the nucleic acid is transferred into the cell and is located, at the end of the process, inside said cell or within or on its membrane. It includes as an essential step crossing of the cellular membrane. However, nucleic acids are not naturally taken up by cells. Accordingly, methods have been proposed permitting this intracellular delivery. This has been achieved by exploiting either the highly sophisticated mechanisms developed by viruses (for a review see Robbins et al., 1998, Tibtech, 16, 35-40) or the use of substances able to bind to nucleic acids in order to form complexes which facilitate introduction of said complexed nucleic acid into cells. These binding substances are principally, while not exclusively, cationic substances which are capable of forming complexes with anionic molecules (widely designated “non-viral synthetic vectors”), thus tending to neutralize the negative charges of nucleic acid allowing to condense it in a complex, and favoring its introduction into the cell. Various methods have been proposed in the literature based on the use of such non-viral synthetic vectors comprising charged substances to improve intracellular uptake of nucleic acids, arguing that these non-viral synthetic vectors present potential advantages with respect to large-scale production, safety, targeting of transfectable cells, low immunogenicity, and the capacity to deliver large fragments of DNA.

In 1989, Felgner et al. (Nature 337, 387-388) proposed the use of cationic lipids which are capable of forming complexes with anionic molecules (i.e. lipoplexes) and favoring their introduction into the cell. Cationic lipids have been used extensively during the last 10 years to facilitate delivery of DNA, mRNA, antisense polynucleotides or proteins into living cells. Since the initial published results, several reagents have become commercially available and additional cationic lipids have been described reporting advantages and widespread utility of these non-viral transfection models. Examples for lipid-mediated transfection substances are DOTMA (Felgner et al., PNAS 84 (1987), 7413-7417), DOGS or Transfectam™ (Behr et al., PNAS 86 (1989), 6982-6986), DMRIE or DORIE (Felgner et al., Methods 5 (1993), 67-75), DC-CHOL (Gao et Huang, BBRC 179 (1991), 280-285), DOTAP™ (McLachlan et al., Gene Therapy 2 (1995), 674-622) or Lipofectamine™, cationic lipids such as described in see WO 98/34910, WO 98137916 or WO 98/56423). Besides, other non-viral delivery systems have been developed which are based on polymer-mediated transfection. There have been many reports on the use for cellular delivery of cationic polymers such as, for example, polyamidoamine (Haensler et Szoka, Bioconjugate Chem. 4 (1993), 372-379), dendrimer polymer (WO 95/24221), polyethylene imine or polypropylene imine (WO 96/02655), polylysine (U.S. Pat. No. 5,595,897 or FR-A-2 719 316). As a general review on these non-viral systems, see Rolland A, Critical reviews in Therapeutic Drug Carrier System, 15, (1998), 143-198.

The exact mechanism governing the non-viral synthetic vectors uptake is still unknown, nevertheless many studies (Felgner et al., 1994, J. Biol. Chem., 269, 2550-2561; Zhou and Huang, 1994, Biochim. Biophys.Acta, 1189, 195-203) indicate that the nucleic acid enters the cell essentially by endocytic uptake of the non-viral synthetic vector. The complex first adsorbs to cell surface by charge interaction and the surface-bound complex is then internalized by endocytosis into endosomes and lysosomes. A small portion of the endocytosed nucleic acid is released into the cytosol from which the nucleic acid, especially DNA, must enter into the nucleus for transcription. The majority of the internalized nucleic acid stays in the endocytic compartments and is eventually degraded. This cellular uptake is a complicated mechanism which involves multiple steps and parameters. One of these critical parameters controlling efficiency of the nucleic acid delivery is the composition of the non-viral synthetic vectors. The complexing substances widely used in the art vary greatly in their chemical structure. For example, cationic substances may contain single or multiple cationic/anionic charges but the overall positive charge must be preserved. Moreover, while most of the above mentioned cationic substances have some level of intrinsic transfection activity alone, it has been shown that addition of additives such as phospholipids, for example phosphatidylethanolamine (PE), can enhance this activity. This improvement is presumably due to their ability to stabilize most type of non-viral synthetic vectors and/or to promote the membrane fusion reaction leading finally to an improved disruption of the endosomal membrane and to an optimal release of the entrapped nucleic acid, and therefore an optimal transfection activity of the carried nucleic acid. This role of PE in membrane fusion has been studied extensively and its mechanism of action can be attributed, at least partially, to its capacity for transition from the bilayer, L alpha, phase into the inverted hexagonal, HII, phase (see also Lasic, 1998, TIBS TECH, 16, 307-321). For example, dioleylphosphatidylethanolamine (DOPE), which on its own is inactive as a transfection reagent presumably due to its inability to spontaneously interact with DNA, has been particularly useful for preparing efficient transfection reagents comprising cationic lipids complexed with plasmid DNA for gene delivery (Felgner et al, 1994, J. Biol. Chem. 269, 2550-2561; Farhood et al., 1995, Biochem Biophys Acta, 1235, 289-295).

Other additives which are thought to improve nucleic acid delivery into cells and to effect the release of said nucleic acid from the endosomes after endocytic uptake by the cells of the nucleic acid/cationic substance complex have been proposed such as those presented below in Table I, or such as steroids (e.g. cholesterol; Templeton et al., 1997, Nat Biotechnol, 15, 647-52).

Nevertheless, while the non-viral synthetic vectors are currently promising, viral vectors despite their major drawbacks in terms of safety are the most useful delivery systems because of their efficiency in transferring genes of interest into cells. It is consequently desirable to ameliorate the non-viral delivery technology especially in order to improve the transfer efficiency of nucleic acids into cell.

WO 90/15807 discloses compounds that are useful as surfactants in the preparation of fluorocarbon emulsions, which can be used as oxygen-carrying blood substitutes, and for therapeutic applications where drugs should be delivered throughout the body, tissue and organs. More specifically, said application discloses the use of particles comprising the discontinuous fluorocarbon phase of the emulsion allowing carrying drugs which are capable to dissolve in fluorocarbon such as for example diazepam, cyclosporin, rifampin, clindamycin, isoflurane, halothane and enflurane or which are capable to complex with, for example, a lecithin membrane such as for example mannitol, tocopherol, streptokinase, dexamethasone, prostaglandin E, Interleukin II, gentamycin and cefoxitin.

Surprisingly, the applicant has now demonstrated that incorporation of a zwitterionic fluorinated compound in a composition comprising a nucleic acid, and preferably at least one substance which binds to a nucleic acid, especially a cationic substance, can greatly enhance the transfer of said nucleic acid into cells.

Thus, the invention relates to composition comprising:

(i) a nucleic acid of interest; and

(iii) a compound, or a combination of compounds, of the general formula:

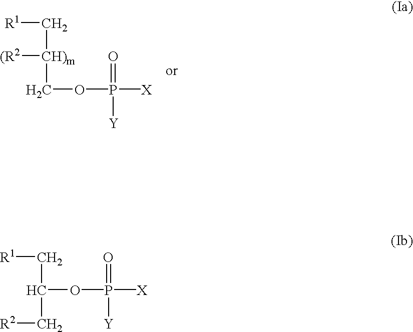

wherein:

R¹ represents:

R_{F}(CH₂)ₐ—(CH═CH)_{b}—(CH₂)_{c}—(CH═CH)_{d}—(CH₂)ₑ—A—;

R_{F}—(CH₂)_{f}—OCH₂CH(CH₂OH)CH₂—A—;

R_{F}—(CH₂)_{g}—OCH₂CH(CH₂OH)—A—;

wherein —A— represents —O—, —C(O), —C(O)O—, —C(S)—, C(O)—S—, —S-, —NH—, —C(O)—NH—, —R⁶(R⁷)N⁺—, (wherein each of R⁶ and R⁷ represents C₁-C₄ alkyl straight or branched chain, or hydroxyethyl), —(CH₂)ₙ—, wherein n=0 or 1, or C(O)N(R⁹)—(CH₂)_{q}—B, wherein q is an integer from 0 to 12, B represents —O—, —C(O), —C(O)O—, —S—, —NH—, —C(S)—, C(O)—S—, —C(O)NH—or —R⁶(R⁷)N⁺—, and wherein a, b, c, d, e, f and g are integers from 0 to 12 where the sum of a+c+e is from 0 to 11, the sum of b+d is from 0 to 12 and each of f and g is from 1 to 12;

R_{F}—(CH₂—CH₂—O)ₕ—;

R_{F}—(CH(CH₃)CH₂O)ₕ—; or

R_{F}(—CH₂—CH₂—S)ₕ—,

wherein h is from 1 to 12; and

wherein R_{F} represents a fluorine-containing moiety having one of the following structures:

(a) F(CF₂)ᵢ—, wherein i is from 2 to 12,

(b) (CF₃)₂CF(CF₂)ⱼ—, wherein j is from 0 to 8,

(c) R_{F}1(CF₂CF(CF₃))ₖ—, wherein k is from 1 to 4, and R_{F}1 represents CF₃—, C₂F₅— or (CF₃)₂CF—,

(d) R_{F}2(R_{F}3)CFO(CF₂CF₂)ₗ—, wherein l is from 1 to 6 and wherein each of R_{F}2 and R_{F}3 independently represents CF₃—, C₂F₅—, n-C₃F₇— or CF₃CF₂CF(CF₃)— or R_{F}2 and R_{F}3 taken together represent —CF₂)₄— or —CF₂)₅—, or

(e) one of the structures (a) to (d) in which one or more of the fluorine atoms are replaced by one or more hydrogen or bromine atoms or hydroxyl group (—OH) and/or at least two chlorine atoms in a proportion such that at least 50% of the atoms bonded to the carbon skeleton of R_{F} are fluorine atoms, and wherein R_{F} contains at least 4 fluorine atoms,

m is 0 or 1;

R² represents R¹, hydrogen or a group —A′—R,

wherein A′ represents —O—, —C(O), —C(O)O—, —C(S)—, C(O)—S—, —S—, —NH—, or —C(O)—NH— and R represents a saturated or unsaturated C₁-C₂₀ alkyl straight chain or branched chain, or C₃-C₂₀ acyl ; and

when m is 1, R¹ and R² may exchange their positions; and

X represents:

—N⁺R⁴R⁵R⁸,

wherein each of R⁴, R⁵ and R⁸ independently represents a hydrogen atom; a C₁-C₄ alkyl group; —CH₂CH₂O(CH₂CH₂O)ₛR³, wherein s represents an integer of from 1 to 5, or R⁴ and R⁵ when taken together represent —(CH2)_{q} wherein q is an integer of from 2 to 5, or when taken together with the nitrogen atom R⁴ and R⁵ form a morpholino group;

—O(CH₂)ₚ—N⁺R⁴R⁵R⁸

wherein R⁴, R⁵ and R⁸ are as defined above, and p is an integer of from 1 to 5; and

Y represents O⁻ or S⁻.

Particularly preferred compounds (iii) in accordance with the invention are those wherein A and/or A′ is —O— (ether group) or —C(O)O— (ester group).

Particularly preferred nucleic acid composition in accordance with the invention is the one wherein said compound (iii) is of the general formula (Ia) presented above and wherein:

m=1;

R¹ is R_{F}(CH₂)ₐ—(CH═CH)_{b}—(CH₂)_{c}—(CH═CH)_{d}—(CH₂)ₑ—A— with a=b=c=0, d=1, e=9, A is —O—, R_{F} is F(CF₂)ᵢ—, and i is 8;

R² is —A′—R, wherein A′ is —O— and R is CH₃—(CH₂)₁₅—;

Y is O⁻;

X is —O(CH₂)ₚ—N⁺R⁴R⁵R⁸ with p=2 and R⁴R⁵ and R⁸ are both hydrogen; and

all other items are as previously indicated (said compound (iii) is referred as pcTG225).

In preferred embodiment, said composition further comprises:

(ii) a substance, or a combination of substances, which binds to a nucleic acid.

Compound (iii) in accordance with the present invention may be prepared by any convenient method, or as disclosed in WO 90/15807 the disclosure of which is specifically incorporated herein by reference in its entirety.

The compositions according to the invention are particularly useful for the introduction or transfer of nucleic acid into cells, e.g. in gene therapy. In a preferred embodiment the composition is a not naturally occurring composition.

The term “nucleic acid” within the present invention is intended to designate any possible nucleic acid, in particular both DNA, RNA or an hybrid form, single or double stranded, linear or circular, natural or synthetic, modified or not (see U.S. Pat. No. 5,525,711, U.S. Pat. No. 4,711,955 or EP-A 302 175 for modification examples). It may be, Inter alia, a genomic DNA, a genomic RNA, a cDNA, an mRNA, an antisense RNA, a ribosomal RNA, a ribozyme, a transfer RNA or DNA encoding such RNAs. The nucleic acid may be in the form of a plasmid or linear nucleic acid which contains at least one expressible sequence that can generate a polypeptide, a ribozyme, an antisense RNA or another molecule of interest upon delivery to a cell. The nucleic acid can also be an oligonucleotide (i.e. a nucleic acid having a short size of less than 100 bp) which is to be delivered to the cell, e.g., for antisense or ribozyme functions. According to the invention, said nucleic acid can be either naked or non-naked. “Naked” means that said nucleic acid, irrespective of its nature (DNA or RNA), its size, its form (single/double stranded, circular/linear, . . . ), is defined as being free from association with transfection-facilitating viral particles, liposomal formulations, charged lipids or polymers and precipitating agents (Wolff et al., Science 247 (1990), 1465-1468; EP 465529). On the opposite, “non-naked” means that said nucleic acid may be associated (i) with viral polypeptides forming what is usually called a virus (adenovirus, retrovirus, poxvirus, etc...) or forming a complex where the nucleic acid while being associated with is not included into a viral element such as viral capsid (see U.S. Pat. No. 5,928,944 and WO 9521259), (ii) with any component which can participate in the transferring uptake of the nucleic acid into the cells with the proviso that the “non-naked” nucleic acid is still negatively charged and/or can still bind to substance (ii). In the case where the nucleic acid is in the form of a virus, composition of the present invention is particularly adapted for masking viral epitope for in vivo applications (with regard to this special issue, see for example the masking approach disclosed in O'Riordan et al., 1999, Human Gene Therapy, 10, 1349-1358). Preferably, the nucleic acid is in the form of plasmid DNA and the polynucleotide is a naked plasmid DNA. A wide range of plasmids is commercially available and well known by one skilled in the art. These available plasmids are easily modified by the molecular biology techniques (see e.g., Sambrook et al, 1989, Laboratory Manual, Cold Spring Harbor Laboratory Press, Cold Spring Harbor, N.Y.). Plasmids derived from pBR322 (Gibco BRL), pUC (Gibco BRL), pBluescript (Stratagene), pREP4, pCEP4 (Invitrogen) and also p Poly (Lathe et al., 1987, Gene 57, 193-201) are illustrative of these modifications. “Nucleic acid” and “polynucleotide” are synonyms.

If the nucleic acid contains the proper genetic information, when it is placed in an environment suitable for gene expression, its transcriptional unit will thus express the encoded gene product. The level of expression will depend to a significant extent on the strength of the associated promoter and the presence and activation of an associated enhancer element. Thus in a preferred embodiment, the transcriptional control element includes the promoter/enhancer sequences such as CMV promoter/enhancer. However, those skilled in the art will recognize that a variety of other promoter and/or enhancer sequences suitable for expression in eukaryotic cells are known and can similarly be used in the delivered encoding nucleic acid. More precisely, these genetic informations necessary for expression by a target cell comprise all the elements required for transcription of said DNA into mRNA and, if necessary, for translation of mRNA into polypeptide. Transcriptional promoters suitable for use in various vertebrate systems are widely described in literature. For example, suitable promoters include viral promoters like RSV, MPSV, SV40, CMV or 7.5k, vaccinia promoter, inducible promoters, etc. The nucleic acid can also include intron sequences, targeting sequences, transport sequences, sequences involved in replication or integration. Said sequences have been reported in the literature and can be readily obtained by those skilled in the art. The nucleic acid or the polynucleotide can also be modified in order to be stabilized with specific components as spermine.

In a preferred embodiment, the nucleic acid contains at least one sequence of interest encoding a gene product which is a therapeutic molecule. A “therapeutic molecule” is one which has a pharmacological or protective activity—when administered appropriately to a patient, especially patient suffering from a disease or illness condition or who should be protected against this disease or condition. Such a pharmacological property is one which is expected to be related to a beneficial effect on the course or a symptom of said disease or said condition. When the skilled man selects in the course of the present invention a gene encoding a therapeutic molecule, he generally relates his choice to results previously obtained and can reasonably expect, without undue experiment other than practicing the invention as claimed, to obtain such pharmacological property. According to the invention, the sequence of interest can be homologous or heterologous to the target cells into which it is introduced. Advantageously said sequence of interest encodes all or part of a polypeptide, especially a therapeutic or prophylactic polypeptide giving a therapeutic or prophylactic property. A polypeptide is understood to be any translational product of a polynucleotide regardless of size, and whether glycosylated or not, and includes peptides and proteins. Therapeutic polypeptides include as a primary example those polypeptides that can compensate for defective or deficient proteins in an animal or human organism, or those that act through toxic effects to limit or remove harmful cells from the body. They can also be immunity conferring polypeptides which act as endogenous immunogens to provoke a humoral or cellular response, or both. Examples of polypeptides encoded by the polynucleotide are enzymes, hornones, cytokines, membrane receptors, structural polypeptides, transport polypeptides, adhesines, ligands, transcription factors, translation factors, replication factors, stabilization factors, antibodies, more especially CFTR, dystrophin, factors VII or IX, E6 or E7 from HPV, MUC1, BRCA1, interferons, interleukin (IL-2, IL-4, IL-6, IL-7, IL-12, GM-CSF (Granulocyte Macrophage Colony Stimulating Factor), the tk gene from Herpes Simplex type 1 virus (HSV-1), p53, suicide polynucleotides (WO 99/54481) or VEGF. The polynucleotide can also code for an antibody. In this regard, antibody encompasses whole immunoglobulins of any class, chimeric antibodies and hybrid antibodies with dual or multiple antigen or epitope specificities, and fragments, such as F(ab)₂, Fab′, Fab including hybrid fragments and anti-idiotypes (U.S. Pat. No. 4,699,880). The nucleic acid sequence of interest encoding a gene product is easily available to those skilled in the art in publications, data bases such as for example GenBank.

According to the invention, “introduction or transfer” means that the nucleic acid is transferred into the cell and is located, at the end of the process, inside said cell or within or on its membrane. It is also called “transfection” or “transduction” depending of the nature of the nucleic acid ; “transfection” is dedicated to design transfer of nucleic acids which do not comprise a viral element such as capsid or viral polypeptides, and “transduction” designates the transfer of viruses. Those terminologies are usual in the technical field of the invention.

According to the present invention, “a substance which binds to a nucleic acid” widely means substances which are able to bind to a nucleic acid, especially those which can further improve the transfer of said nucleic acid into cells because of this binding, irrespective of the nature of the binding. More particularly, this binding can be mediated by hydrostatic, hydrophobic, cationic, covalent or non covalent bonds.

In a preferred embodiment, this substance is selected from the group consisting of chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile or derivatives (see EP 890 362), cytokines, especially interleukin-10 (IL-10) (PCT/EP/99 03082), hyaluronidase (WO 98/53853) and nuclease inhibitors (PCT/EP/99 03082) such as, for example, actin G.

In another embodiment, this substance can be a in salt, and preferably a cationic salt such as magnesium (Mg²⁺) (EP 9911957.0) and/or lithium (Li⁺). In this case, the amount of ionic substance in the composition of the invention preferably ranges from about 0.1 mM to about 100 mM, and still more preferably from about 0.1 mM to about 10 mM.

In a further preferred embodiment, this substance can encapsulate the nucleic acid (i). One particularly attractive example with this respect are the nanoparticles provided by binding of said nucleic acid with special polymers such as for example poly(lactide-co-glycolide), biodegradable or poly(lactide)-poly(ethylene glycol) (Hawley et al., 1997, Pharm Res. 14, 657-661 ; Hedley et al., 1998, Nat. Med., 4, 365-368).

In a preferred embodiment, the composition according to the invention comprises at least one substance (ii) which is a cationic substance, and in a still more preferred embodiment said cationic substance is a cationic lipid or a cationic polymer. The composition can also comprise a mixture of various substances (ii).

Examples of cationic lipids or cationic polymers are provided above in the specification. Advantageously, said cationic lipids are selected from among cationic lipids of the formula (see WO 98/34910):

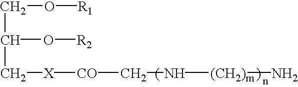

wherein:

R₁, R₂, are identical or different and are C₆-C₂₃ alkyl or alkenyl, linear or branched, or —C(═O)—(C₆-C₂₃)alkyl or —C(═O)—(C₆-C₂₃)alkenyl, linear or branched,

X is O, S, S(O) or —NR₃, R₃ being H or C₁-C₄alkyl,

n=1 to 6,

m=1 to 6, and when n>1, m can be identical or different.

According to another preferred embodiment, the cationic lipid is selected from cationic lipids of the following formula:

R—HN—[—(CH₂)ₘ—NR—]ₙ₋₁—(CH₂)ₘ—NH—R

wherein:

R is, independently of one another, H or

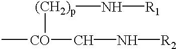

wherein:

R₁ and R₂ are, independently of one another C₆-C₂₃ alkyl or alkenyl, linear or branched, or —C(═O)—(C₆-C₂₃) alkyl or —C(═O)—(C₆-C₂₃) alkenyl, linear or branched, aryl, cycloalkyl, fluoroalkyl, polyethylene glycol, oxyethylene ou oxymethylene radicals,

p=1 to 4,

n=1 to 6,

m=1 to 6.

Cationic polymers or mixtures of cationic polymers which may be used in the present invention include cationic polymers selected from the group consisting of chitosan, poly(aminoacids) such as polylysine (U.S. Pat. No. 5,595,897 and FR 2 719 316); polyquaternary compounds; protamine; polyimines; polyethylene imine or polypropylene imine (WO 96/02655); polyvinylamines; polycationic polymer derivatized with DEAE, such as pullulans, celluloses; polyvinylpyridine; polymethacrylates; polyacrylates; polyoxethanes; polythiodiethylaminomethylethylene (P(TDAE)); polyhistidine; polyomithine; poly-p-aminostyrene; polyoxethanes; co-polymethacrylates (eg copolymer of HPMA; N-(2-hydroxypropyl)-methacrylamide); the compound disclosed in U.S. Pat. No. 3,910,862, polyvinylpyrrolid complexes of DEAE with methacrylate, dextran, acrylamide, polyimines, albumin, onedimethylaminomethylmethacrylates and polyvinylpyrrolidonemethylacrylaminopropyltrimethyl ammonium chlorides; polyamidoamines; telomeric compounds (patent application filing number EP (98401471.2). Nevertheless, this list is not exhaustive and other cationic polymers well known in the art can be used in the composition according to the invention as well. Additionally, those cationic lipids and cationic polymers might be themselves fluorinated (see WO 98/34910 for example).

According to a particularly preferred embodiment, the cationic polymer is a substituted polyvinylamine such as defined by formula:

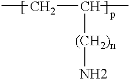

wherein n=0 to 5, p=2 to 20000

wherein:

at least 10% of free NH₂ are substituted with R, and R is an hydrophilic group.

According to another preferred embodiment said cationic polymer is a polymer of the general formula:

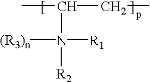

wherein:

the degree of polymerization p ranges from 2 to 1000000;

R₁, R₂ and R₃, independently of one another in each [CH—CH₂] repeat, are selected from H, alkyl of 1 to 20 carbon atoms or aryl of 5 to 7 carbon atoms; n is 0 or 1, with the proviso that at least one n is 1 in the full length of the polymer.

In a further preferred embodiment the composition according to the invention further comprises:

(iv) at least one additive which is selected from the group consisting of neutral, zwitterionic and negatively-charged lipids.

Such neutral, zwifterionic and negatively charged lipids can ,e.g., be selected from the group consisting of natural or synthetic components:

natural phospholipids which are typically from animal and plant sources, such as phosphatidylcholine, phosphocholine, phosphatidylethanolamine, sphingomyelin, phosphatidylserine, or phosphatidylinositol, ceramide or cerebroside and their analogs;

synthetic phospholipids which are typically those having identical fatty acid groups, including, but not limited to, dimyristoylphosphatidylcholine, dioleoylphosphatidylcholine, dipalmitoylphosphatidylcholine, distearoylphosphatidylcholine, phosphatidylethanolamine (PE) and phosphatidylglycerol, and their analogues;

more specifically, the neutral lipid can, e.g., be phosphatidylcholine, cardiolipin, phosphatidylethanolamine, mono-, di- or triacylglycerols, or analogues thereof;

the negatively charged lipid can, e.g., be phosphatidylglycerol, phosphatidic acid or a similar phospholipid analog;

other additives such as cholestrerol, glycolipids, fatty acids, sphingolipids, prostaglandins, gangliosids, niosomes, or any other natural or synthetic amphiphiles can also be used in formulation of the present invention, as is conventionally known in the art.

Among preferred additives (iv) are analogs of the phosphatidylethanolamines (PE), such for example those presented in following Table I:

Neutral Lipid Side Chain Variations

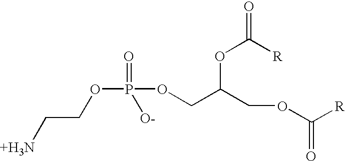

| | |
|---|---|
| DMPE | R = (CH₂)₁₂CH₃ |
| DPPE | R = (CH₂)₁₄CH₃ |
| DSPE | R = (CH₂)₁₆CH₃ |

Neutral Lipid Amine Lipids

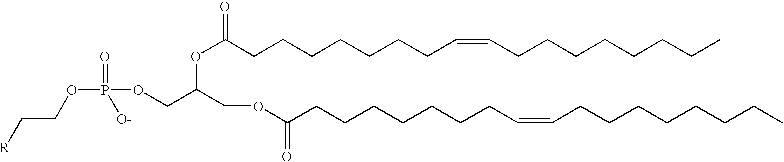

| | | | |
|---|---|---|---|
| DOPE | R = H₃N+ | DOPC | R = (CH₃)₃N+ |
| PMME | R = CH₃H₂N+ | CPE | R = H₃N+(CH₂)₆ |
| PDME | R = (CH₃)₂HN+ | DPE | R = H₃N+(CH₂)₁₂ |

Mono-acyl Neutral Lipids

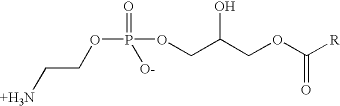

| | |
|---|---|
| lysoOPE | R = (CH₂)₇[Z]CH═CH(CH₂)₇CH₃ |
| lysoMPE | R = (CH₂)₁₂)CH₃ |
| lysoPPE | R = (CH₂)₁₄CH₃ |
| lysoSPE | R = (CH₂)₁₆CH₃ |

In a particularly preferred embodiment said additive (iv) is the dioleoylphosphatidylethanolamine (DOPE).

The non naturally occurring nucleic acid compositions of the invention can further be characterized by independent factors.

In a first aspect, said compositions may be characterized by their theoretical charge ratio (+/−), which is the ratio of:

the number of positive charges provided by a first group including at least the substance (ii) where it is a cationic substance, the compound (iii), and optionally the additive (iv), or combination of such substances, compounds and/or additives,

to the number of negative charges provided by a second group including at least the nucleic acid (i) in the composition,

assuming that all potentially cationic groups are in fact in the cationic state and all potentially anionic groups are in fact in the anionic state. In general, an excess of positive charges on the composition facilitates binding of the composition to the negatively-charged cell surface. To obtain such a ratio, the calculation shall take into account all negative charges provided by said second group and shall then adjust the quantity of substance (ii), compound (iii), and optionally the additive (iv), necessary to obtain the desired theoretical charge ratio indicated above. The quantities and the concentrations of all ingredients shall be adjusted in function of their respective molar masses and their number of positive charges. The ratio is not specifically limited. Quantities are selected so that the ratio between the number of positive charges and the number of negative charges varies from between 0.05 and 20, preferably between 0.1 and 15, and most preferably around 0.5 to 10.

In a second aspect, said compositions may be characterized by the concentration of the nucleic acid (i) which preferably ranges from 10 μg/ml to 5000 μg/ml. In preferred embodiments of the invention, the concentration of said nucleic acid ranges from 100 μg/ml to 2000 μg/ml. Additionally, the form of the nucleic acid can affect the expression efficiency, and it is preferable that a large fraction of the nucleic acid be in supercoiled form. Thus, in a preferred embodiment, at least 80, more preferably at least 90 and most preferably at least 95% of the nucleic acid in the composition is supercoiled.

In a third aspect, the composition may be characterized by the ratios of substance (ii) to compound (iii) (on a molar basis) which preferably varies from between 0.1 and 20, preferably between 0.3 and 10, and most preferably around 0.5 to 5.

In a fourth aspect, said compositions may be characterized by the ratios of substance (ii) to additive (iv) (on a molar basis) when the two types are co-existing in the composition. This ratio preferably ranges from between 0.1 and 10, more preferably between 1 and 10, and most preferably around 2 to 5.

In a preferred embodiment, the molar ratio between said substance (ii) said compound (iii)/and said additive (iv) in the composition of the present invention varies from 1/0.05/0 to 1/10/4, preferably from 1/2/1 to 1/4/2 and still more preferably is 1/1.5/0.5.

In a fifth aspect, said compositions may be characterized by the average diameter of the composition according to the invention which is small (preferably less than 2 μm). In a preferred embodiment, this average diameter is between about 20 and 800 nm, more preferably between about 50 and 500 nm, still more preferably between about 75 and 200 nm, and most preferably about 100 nm. A composition average diameter may be selected for optimal use in particular applications. Measurements of the composition average diameter can be achieved by a number of techniques including, but not limited to, dynamic laser light scattering (photon correlation spectroscopy, PCS), as well as other techniques known to those skilled in the art (see, Washington, Particle Size Analysis in Pharmaceutics and other Industries, Ellis Horwood, New York, 1992, 135-169). Sizing procedure may also be applied on compositions in order to select a specific composition diameter. Methods which can be used in this sizing step include, but are not limited to, extrusion, sonication and microfluidization, size exclusion chromatography, field flow fractionation, electrophoresis and ultracentrifugation. In a preferred embodiment, the composition is prepared in an aqueous carbohydrate solution which is approximately isotonic with animal cells. More preferably, the carbohydrate is lactose or glucose, and is present in amount varying around 5 to 10%.

Furthermore, due to the presence of reactive functions (amino, hydroxy, etc . . . ) in either nucleic acid (i), substance (ii), compound (iii) and/or additive (iv), all or part of the composition can be substituted, directly or via a spacer such as heterobifunctional reactives such as SPDP or SMCC, or functionalized PEG which are well known by the person skilled in the art (Maftson et al., 1993, Mol. Biol. Reports, 17, 167-183). The substituent can be at least one element of those widely disclosed in scientific publications, e.g., labelling molecules (see, for example, molecules disclosed in U.S. Pat. No. 4,711,955) enabling, for example, visualization of the distribution of the composition after in vitro or in vivo administration; cell targeting molecules (ligands) or anchoring molecules; elements facilitating penetration into the cell, lysis of endosomes (JTS1 peptides for example, Gottchalk et al., 1996, Gene Therapy, 3, 448-457) or even intracellular transport towards the nucleus. These elements may be composed of all or part of sugars, glycol, peptides (e.g. GRP, Gastrin Releasing Peptide), oligonucleotides, lipids (especially those with C2-C22, hormones, vitamins, antigens, antibodies (or fragments thereof), specific membrane receptor ligands, ligands capable of reaction with an anti-ligand, fusogenic peptides, nuclear localization peptides, or a combination of said compounds, e.g. galactosyl residues to target the asialoglycoprotein receptor on the surface of hepatocytes, the INF-7 fusogenic peptide derived from the HA-2 subunit of the influenza virus hemagglutinin (Plank et al. 1994, J. Biol. Chem. 269, 12918-12924) for membrane fusion, or a nuclear signal sequence derived from the T-antigen of the SV40 virus (Lanford and Butel, 1984, Cell 37, 801-813) or from the EBNA-1 protein of the Epstein Barr virus (Ambinder et al., 1991, J. Virol. 65, 1466-1478). Furthermore, the reactive groups can be substituted with alkyl C1-C6, leading for example to permethylated compositions. The reactive groups might also be substituted with amino groups. Such substituted nucleic acid (i), substance (ii), compound (iii) and/or additive (iv), can be obtained easily using the techniques described in the literature, especially by chemical coupling, notably by using protective groups such as trifluoroacetyl, Fmoc (9-fluorenylmethoxycarbonyl) or BOC (tert-butyl oxycarbonyl) on the amine moiety. Selective removal of a protective group then allows coupling of the targeting element, and then complete deprotection of the targeted component (Greene T. W. and Wuts P. G. M., 1991, Protective groups in organic synthesis. Ed. J. Wiley & Sons, Inc. New York).

The invention also relates to a process for preparing the claimed compositions, said process comprising the steps of bringing one or more nucleic acid (i), one or more substance (ii), one or more compounds (iii), and optionally one or more additive (iv) into contact and of recovering the composition, optionally after a purification and/or sizing step.

In a first variant, one or more substances (ii), i.e. cationic lipids, one or more compounds (iii), and optionally one or more additives (iv) are dissolved in an appropriate organic solvent such as chloroform. The mixture is then dried under vaccum. The film obtained is further dissolved in an appropriate amount of solvent or mixture of solvents which are miscible in water, in particular ethanol, dimethylsulfoxide (DMSO), or preferably a 1:1 (v:v) ethanol : DMSO mixture, so as to form lipid aggregates according to a known method (WO 96/03977), or in a second variant, are suspended in an appropriate quantity of a solution of detergent such as an octylglucoside (e.g. n-octyl-beta-D-glucopyranoside or 6-O-(N-heptylcarbamoyl)-methyl-alpha-D-glucopyranoside).

The suspension may then be mixed with a solution comprising the desired amount of nucleic acid (i).

In the case of the second variant and optionally, subsequent dialysis may be carried out in order to remove the detergent and to recover the composition of the invention. The principle of such a method is described by Hofland et al., 1996 (Proc. Natl. Acad. Sci., 93 7305-7309).

According to a third variant, one or more substance (ii), one or more compound (iii), and optionally one or more additive (iv) are suspended in a buffer and then the suspension is subjected to sonication until visual homogeneity is obtained. The lipid suspension is then extruded through two microporous membranes under appropriate pressure. The lipid suspension is then mixed with a solution of nucleic acid (i). This so-called sonication-extrusion technique is well known by those skilled in the art.

The characteristics of the compositions formed may be evaluated by several means which make it possible to determine, for example:

the state of the composition formation with the nucleic acid, in particular by identification of the free nucleic acids by agarose gel electrophoresis,

the size of the composition by a quasi elastic scattering of light,

the absence of precipitation over the long term.

The invention also relates to a formulation for the transfection of a nucleic acid into cells, comprising at least one composition according to the invention. This formulation can be in various forms, e.g. in solid, liquid, powder, aqueous, lyophilized form. In a preferred embodiment, this formulation further comprises a pharmaceutically acceptable carrier, allowing its use in a method for the therapeutic treatment of humans or animals. In this particular case, the carrier is preferably a pharmaceutically suitable injectable carrier or diluent (for examples, see Remington's Pharmaceutical Sciences, 16^{th} ed. 1980, Mack Publishing Co). Such carrier or diluent is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers or adjuvants. The pH of the pharmaceutical preparation is suitably adjusted and buffered in order to be useful in in vivo applications. It may be prepared either as a liquid solution or as a solid form (e.g. lyophilized) which can be suspended in a solution prior to administration. Representative examples of carriers or diluents for an injectable formulation include water, isotonic saline solutions which are preferably buffered at a physiological pH (such as phosphate buffered saline or Tris buffered saline), mannitol, dextrose, glycerol and ethanol, as well as polypeptides or proteins such as human serum albumin. For example, such formulations comprise a composition of the invention in 10 mg/ml mannitol, 1 mg/ml HSA, 20 mM Tris pH 7.2 and 150 mM NaCl.

The present invention also relates to a method for introducing a nucleic acid into a cell wherein said method comprises the step of contacting a cell with a composition or formulation according to the invention, whereby said nucleic acid (i) is taken up into said cell.

This method may be applied by direct administration of said nucleic acid composition or said formulation to cells of the animal in vivo, or by in vitro treatment of cells which were recovered from the animal and then re-introduced into the animal body (ex vivo process). In in vitro application, cells cultivated on an appropriate medium are placed in contact with said nucleic acid composition or said formulation. After an incubation time, the cells are washed and recovered. Introduction of the active substance can be verified (eventually after lysis of the cells) by any appropriate method.

To the same extent, the present invention relates to a method for treatment of a mammal suffering from a disease or illness condition, or who should be protected against this disease or condition, comprising the steps of:

(a) administering to a mammal a therapeutically effective amount of a composition or of a formulation according to the invention, wherein said nucleic acid (i) is specific for-the treatment of said condition or said disease, and

(b) permitting said nucleic to be incorporated into at least one cell of said patient whereby said disease is effectively treated.

In a preferred embodiment of this method a formulation according to the invention is used which comprises a pharmaceutically acceptable carrier.

According to the invention, “cells” means both prokaryotic cells and eukaryotic cells, yeast cells, plant cells, human or animal cells, in particular mammalian cells. In particular, cancer cells should be mentioned. The term “cells” should be understood broadly without any limitation concerning particular organization in tissue, organ, etc. To the same extent, it should be understood as meaning isolated cells.

The methods according to the invention can be applied in vivo, e.g., to the interstitial or luminal space of tissues in the lungs, the trachea, the skin, the muscles, the brain, the liver, the heart, the spleen, the bone marrow, the thymus, the bladder, the lymphatic system, the blood, the pancreas, the stomach, the kidneys, the ovaries, the testicles, the rectum, the peripheral or central nervous system, the eyes, the lymphoid organs, the cartilage or the endothelium. The composition or formulation can,e.g., be administered into target tissues of the vertebrate body including those of muscle, skin, brain, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor, etc.

The composition or formulation of the present invention can be administered, e.g., by intradermal, subdermal, intravenous, intramuscular, intranasal, intracerebral, intratracheal, intraarterial, intraperitoneal, intravesical, intrapleural, Intracoronary or intratumoral injection, by means of a syringe or other devices. Transdermal administration is also contemplated, as are inhalation, aerosol routes, instillation or topical application.

Applied to in vivo gene therapy, the present invention allows repeated administration to the patient without any risk of the administered preparation to induce a significant immune reaction. Administration may be by single or repeated dose, once or several times after a certain period of time. Repeated administration allows a reduction of the dose of nucleic acid administered at a single time. The route of administration and the appropriate dose varies depending on several parameters, for example the individual patient, the disease being treated, or the nucleic acid being transferred.

In the case of in vivo treatment according to the invention, in order to improve the transfection rate, the patient may undergo a macrophage depletion treatment prior to administration of the pharmaceutical preparations described above. Such a technique is described in the literature (see, e.g., Van Rooijen et al., 1997, TibTech, 15, 178-184).

In a preferred embodiment, the administration method can be advantageously Improved by combining injection in an afferent and/or efferent fluid vessel with an increase of permeability of said vessel. Preferably, said increase is obtained by increasing hydrostatic pressure (e.g. by obstructing outflow and/or inflow), osmotic pressure (with hypertonic solution) and/or introducing a biologically-active molecule (e.g. histamine into the administered composition) (see WO 98/58542).

The concentration of the nucleic acid in the composition or formulation is preferably from about 0.01 mM to about 1 M, and more preferably from about 0.1 mM to 10 mM.

The present invention also relates to the use of the composition of the invention for the transfer of a nucleic acid into a cell, either in vitro (or ex vivo, see above) or in vivo. Preferably, it relates to the use of the composition of the invention for improving the transfer of a nucleic acid into a cell. “Improving transfer of a nucleic acid into a cell” means, in this regard, a more efficient transfer of a said nucleic acid by cells when such composition is used compared to an introduction performed without such a composition. This can be determined by comparing the amount of the nucleic acid taken up with another composition and comparing this amount with the amount taken up by the cells when using the composition of the invention under the same experimental conditions. Preferably, the improved transfer can be determined by a higher amount of expression of the nucleic acid transferred into the cells when using the composition of the invention in comparison to a situation where another composition is used.

Thus, the present invention further relates to the use of the composition of the invention as an active pharmaceutical substance.

Finally, the present invention concerns the use of the composition of the invention for the preparation of a pharmaceutical formulation for the introduction of a nucleic acid into cells. It was surprisingly found that the use of the composition according to the invention for transferring a nucleic acid into vertebrate cells, leads to a dramatic improvement of the transfer efficiency. Thus, the present invention preferably relates to the use of the composition of the invention for the preparation of a pharmaceutical composition for an improved transfer of a nucleic acid into a cell.

The present invention also relates to the use of a compound (iii) as defined herein above for the transfer of a nucleic acid into a cell as well as to the use of a compound (iii) as defined herein above for the preparation of a composition for introducing a nucleic acid into cell.

The methods, compositions and uses of the invention can be applied in the treatment of all kinds of diseases the treatment and/or diagnostic of which is related to or dependent on the transfer of nucleic acids in cells. The compositions, methods and uses of the present invention may be desirably employed in humans, although animal treatment is also encompassed by the methods and uses described herein.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nlm.nih.gov, http://www.infobiogen.fr, http://www.fmi.ch/biology/research_tools.html, http://www.tigr.org, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced different from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGENDS OF THE FIGURES

FIG. 1: Expression of luciferase in A549 cells transfected in vitro with different amounts of plasmid DNA and different pcTG90/pcTG225/DOPE ratios at N/P=10. Transfection was carried out in the absence and presence of 10% fetal calf serum. Expression was stopped 48 hours after transfection.

FIG. 2: In vivo expression of luciferase in lungs 24 hours after injection of different pcTG90/pcTG225/DOPE ratios at N/P 10.

FIG. 3: In vivo expression of luciferase in lungs 24 hours after injection of different pcTG90/pcTG225/DOPE ratios at N/P 5.

### COMPOSITION FORMULATIONS

The desired amounts of each lipid in chloroform were mixed in the mentioned molar ratio (see legend). Chloroform was then evaporated under vacuum for 2 hr at 45° C. (Laconco, Rotavap, Uniequip, Munich, Germany) and the dried lipid films were hydrated in 5% glucose. The resulting lipid mixtures were sonicated for 20 min. Compositions of the desired cationic lipid pcTG90/nucleic acid ratio (N/P ratio; Zanta et al., (1997) Gene Therapy 8, 839-844) were prepared the day before injection by adding the cationic substance to the desired amount of plasmid diluted in 5% glucose and stored at 4° C. The exact structure of pcTG90 is disclosed in EP 901463.

### Example of Preparation: pcTG90/pcTG225/DOPE 1:1.5:0.5 at N/P 10

420 μg plasmid DNA (1 μg DNA/μl) corresponds to 1.273 μmoles negatively charged phosphate (molecular weight of one base=330 Da). To get N/P 10, one needs 12.727 μmoles amino group. Assuming that pcTG90 is totally protonated at pH 7.4 (1 mole pcTG90 corresponds to 5 moles amino group), one needs 2.5454 μmoles pcTG90 (3.754 mg). For preparation of liposomes pcTG90/pcTG225/DOPE 1:1.5:0.5, one needs 3.854 mg pcTG225 and 0.947 mg DOPE. pcTG90, pcTG225 and DOPE were dissolved in chloroform which was then evaporated under vacuum for 2 hr at 45° C. (Laconco, Rotavap, Uniequip, Munich, Germany) and the dried lipid films were hydrated in 375.4 μl, 5% glucose and then sonicated for 20 min. Total volume of formed liposomes was added to the DNA solution (420 μg+344 μl 20% glucose (w/v)+611 μl mQ water). The preparation is stored at 4° C. until it is used.

### Efficiency of the Transfection of A549 Cells

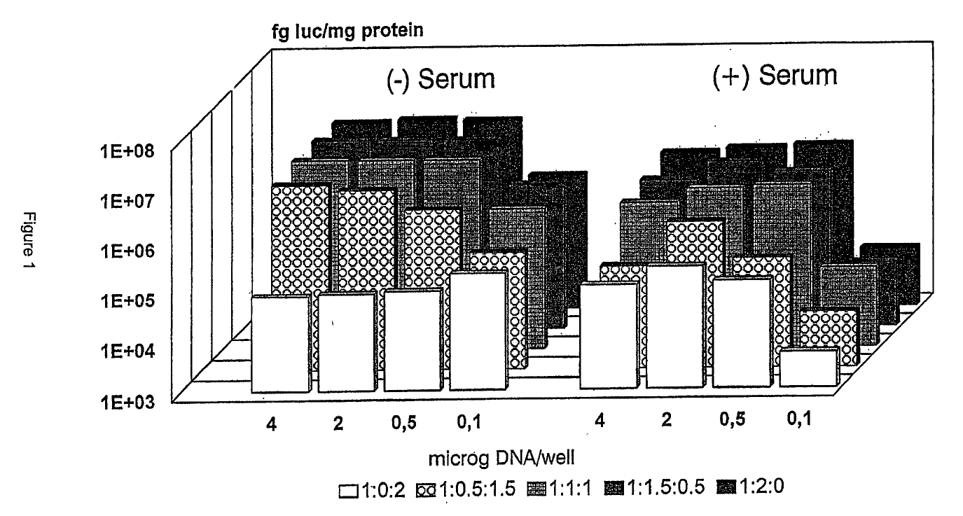

Twenty-four hours before transfection, A549 cells (epithelial cells derived from human pulmonary carcinoma) were cultivated in Dulbeco-modified Eagle culture medium (DMEM), containing 10% fetal calf serum (Gibco BRL), in 96 multi-wells plates wells (2×10⁴ cells per well), in a humid (37° C.) and 5% CO₂/95% air atmosphere. Volume of preparations at 0.1 mg/ml plasmid DNA (40, 20, 5 and 1 μl, respectively) was diluted to 100 μl in DMEM or DMEM supplemented with 10% fetal calf serum (for transfection performed in the presence of serum) in order to obtain various amounts of DNA (4, 2, 0.5 and 0.1 μg, respectively) in the preparation. The culture medium was removed and replaced by 100 μl of DMEM with or without 10% serum containing the desired amount of DNA. 50 μl of DMEM+30% fetal calf serum (or 10% for the transfections made with serum) were added. After 20 hours, 100 μl of DMEM+10% serum were added. 48 hours after transfection, the culture medium was discarded and the cells were washed with 100 μl of phosphate solution PBS and Lysol with 50 μl of lysis buffer (Promega). The lysates were frozen at −80° C. awaiting analysis of luciferase activity. This measurement was done for 15 seconds on 20 μl of the lysis mixture in a Berthold LB96P luminometer, using the Luciferase determination assay (Promega) in 96-well plates. The results are illustrated in FIG. 1. The values are expressed in fg luciferase per mg of protein. The total protein concentration per well was determined using conventional techniques (BCA test, Pierce).

These results indicate that the transfection efficiency increases with increase of the amount of pcTG225, to reach a maximum at pcTG90/pcTG225/DOPE ratio 1:0.5:1.5. pcTG90/pcTG225 (1:2) preparations were still approximately 2 times more efficient than preparations pcTG90/DOPE (1:2). When transfections were performed in the presence of serum, the same pattern is obtained showing that the pcTG225 effect is not hampered by the serum (FIG. 1).

### Composition Injection

9 week-old female B6SJLF1 mice (Iffa-Credo, l'Arbresle, France) were injected intravenously into the tail vein with 250 μl (60 μg DNA) of the desired composition. 24 hours later mice were sacrificed and lungs removed and frozen in liquid nitrogen. Determination of luciferase expression was performed according to the protocol described by Schughart et al. (Gene Therapy 6 (1999), 448-453). Tissues were disrupted in 500 μl of lysis buffer (Promega, Charnonnieres, France) with a homogenizer (two 30 sec pulses in a Polytron homogenizer; Kinematica, Littau, Switzeland). The homogenates were then freeze-thawed three times and cells debris removed by centrifugation. 20 μl of the supernatant were used to determine luciferase activity (luminometer Microlumat LB 96P; Berthold, Evry, France). Proteins were quantified by bicinchoninic acid (BCA) protein assay (Pierce, Montiuron, France). Results are given as relative light units (RLU) per min per mg protein.

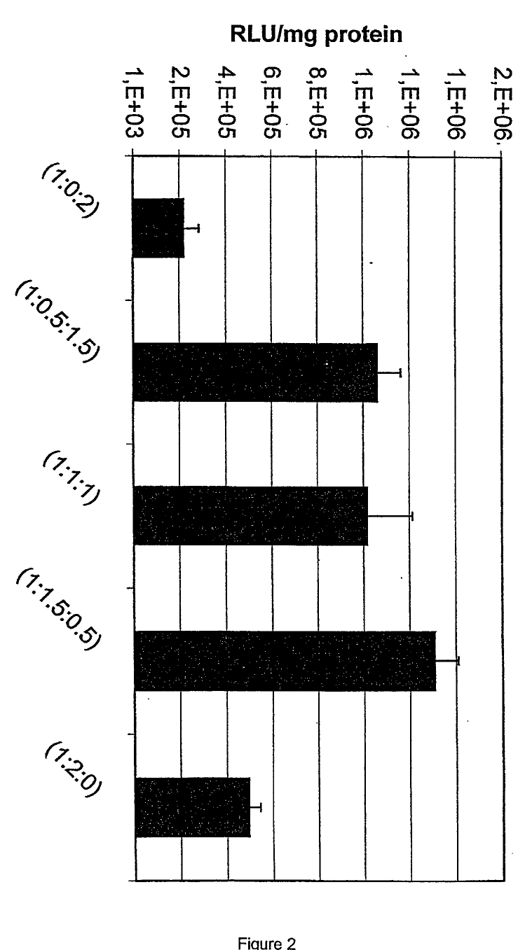

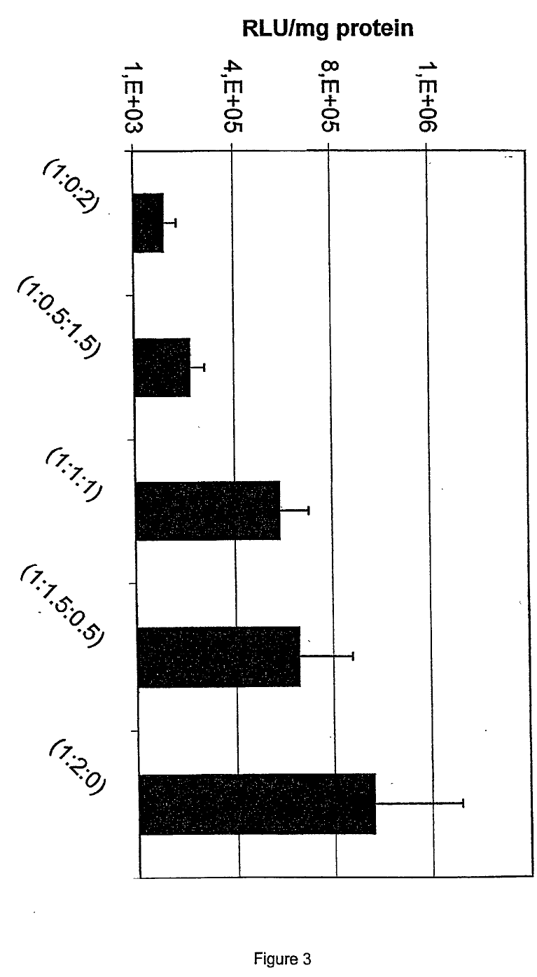

The in vivo results (FIGS. 2 and 3) are consistent with the in vitro ones. They show an increase of efficiency when pcTG225 is incorporated in the preparations, whatever the charge ratio used. At N/P 10 (FIG. 2), as the amount of pcTG225 increases transfection efficiency increases to reach a plateau at pcTG90/pcTG225/DOPE ratio 1:1.5:0.5. pcTG90/pcTG225 (1:2) compositions are approximately 2.5 times more efficient than pcTG90/DOPE (1:2). When compositions are used at N/P 5 (FIG. 3), transgene expression efficiency increases with the amount of pcTG225 achieving levels of expression approx. 8 times higher than those with pcTG90/DOPE (1:2), 4.4 times higher than pcTG90/DOPE (1:2) N/P 10 and similar to those obtained with preparations containing pcTG225 at N/P 10.

What is claimed is:

1. A composition comprising:
(i) a nucleic acid of interest; and
(iii) a compound, or a combination of compounds, of the general formula: 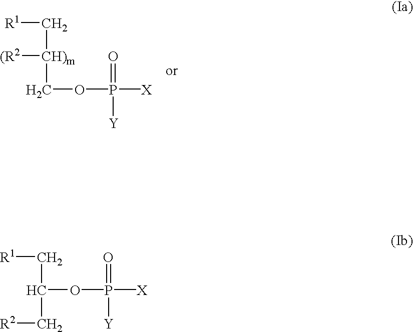
wherein:
R¹ represents:
R_{F}(CH₂)ₐ—(CH═CH)_{b}—(CH₂)_{c}—(CH═CH)_{d}—(CH₂)ₑ—A—;
R_{F}—(CH₂)_{f}—OCH₂CH(CH₂OH)CH₂—A—;
R_{F}—(CH₂)_{g}—OCH₂CH(CH₂OH)—A—;
wherein —A— represents —O—, —C(O), —C(O)O—, —C(S)—, C(O)—S—, —S-, —NH—, —C(O)—NH—, —R⁶(R⁷)N⁺—, (wherein each of R⁶ and R⁷ represents C₁-C₄ alkyl straight or branched chain, or hydroxyethyl), —(CH₂)ₙ—, wherein n=0 or 1, or C(O)N(R⁹)—(CH₂)_{q}—B, wherein q is an integer from 0 to 12, B represents —O—, —C(O), —C(O)O—, —S—, —NH—, —C(S)—, C(O)—S—, —C(O)NH—or —R⁶(R⁷)N⁺—, and wherein a, b, c, d, e, f and g are integers from 0 to 12 where the sum of a+c+e is from 0 to 11, the sum of b+d is from 0 to 12 and each of f and g is from 1 to 12;
R_{F}—(CH₂—CH₂—O)ₕ—;
R_{F}—(CH(CH₃)CH₂O)ₕ—; or
R_{F}(—CH₂—CH₂—S)ₕ—,
wherein h is from 1 to 12; and
wherein R_{F} represents a fluorine-containing moiety having one of the following structures:
(f) F(CF₂)ᵢ—, wherein i is from 2 to 12,
(g) (CF₃)₂CF(CF₂)ⱼ—, wherein j is from 0 to 8,
(h) R_{F}1(CF₂CF(CF₃))ₖ—, wherein k is from 1 to 4, and R_{F}1 represents CF₃—, C₂F₅— or (CF₃)₂CF—,
(i) R_{F}2(R_{F}3)CFO(CF₂CF₂)ₗ—, wherein l is from 1 to 6 and wherein each of R_{F}2 and R_{F}3 independently represents CF₃—, C₂F₅—, n-C₃F₇— or CF₃CF₂CF(CF₃)— or R_{F}2 and R_{F}3 taken together represent —CF₂)₄— or —CF₂)₅—, or
(j) one of the structures (a) to (d) in which one or more of the fluorine atoms are replaced by one or more hydrogen or bromine atoms or hydroxyl group (—OH) and/or at least two chlorine atoms in a proportion such that at least 50% of the atoms bonded to the carbon skeleton of R_{F} are fluorine atoms, and wherein R_{F} contains at least 4 fluorine atoms,
m is 0 or 1;
R² represents R¹, hydrogen or a group —A′—R,
wherein A′ represents —O—, —C(O), —C(O)O—, —C(S)—, C(O)—S—, —S—, —NH—, or —C(O)—NH— and R represents a saturated or unsaturated C₁-C₂₀ alkyl straight chain or branched chain, or C₃-C₂₀ acyl ; and
when m is 1, R¹ and R² may exchange their positions; and
X represents:
—N⁺R⁴R⁵R⁸,
wherein each of R⁴, R⁵ and R⁸ independently represents a hydrogen atom; a C₁-C₄ alkyl group; —CH₂CH₂O(CH₂CH₂O)ₛR³, wherein s represents an integer of from 1 to 5, or R⁴ and R⁵ when taken together represent —(CH2)_{q} wherein q is an integer of from 2 to 5, or when taken together with the nitrogen atom R⁴ and R⁵ form a morpholino group;
—O(CH₂)ₚ—N⁺R⁴R⁵R⁸
wherein R⁴, R⁵ and R⁸ are as defined above, and p is an integer of from 1 to 5; and
Y represents O⁻ or S⁻.

2. The composition of claim 1, wherein said compound (iii) is of the general formula (Ia) wherein :
m=1;
R¹ is R_{F}(CH₂)ₐ—(CH═CH)_{b}—(CH₂)_{c}—(CH═CH)_{d}—(CH₂)ₑ—A— with a=b=c=0, d=1, e=9, A is —O—, R_{F} is F(CF₂)ᵢ—, and i is 8;
R² is —A′—R, wherein A′ is —O— and R is CH₃—(CH₂)₁₅—;
Y is O⁻;
X is —O(CH₂)ₚ—N⁺R⁴R⁵R⁸ with p=2 and R⁴R⁵ and R⁸ are both hydrogen.

3. The composition of claim 1 or 2, wherein it further comprises (ii) a substance, or a combination of substances, which binds to a nucleic acid.

4. The composition of claim 3 wherein said substance (ii) which binds to a nucleic acid is a cationic substance.

5. The composition of claim 4 wherein said cationic substance is a cationic lipid or a cationic polymer.

6. The composition of anyone of claims 1 to 5, wherein said nucleic acid composition further comprises:
(iv) at least one additive which is selected from the group consisting of neutral, zwitterionic and negatively charged lipids.

7. The composition of claim 6, wherein said additive (iv) is the dioleoylphosphatidylethanolamine (DOPE).

8. The composition of anyone of claims 1 to 7, wherein said substance (ii)/compound (iii) molar ratio is between 0.1 and 10.

9. The composition of claim 5 or 6, wherein the molar ratio between said substance (ii)/said additive (iv)/and said compound (iii) is from 1/0.05/0 and 1/10/4.

10. The composition of anyone of claims 1 to 6, wherein the ratio between:
the number of positive charges of a first group including the compound (iii), the substance (ii) where it is a cationic substance and optionally the additive (iv), and
the number of negative charges of a second group including at least said nucleic acid (i), varies from 0.05 to 20.

11. The composition of one of claims 1 to 10, wherein said composition has a diameter of between about 20 and 800 nm.

12. A formulation for the transfection of a nucleic acid into cells, comprising the composition of anyone of claims 1 to 10.

13. The formulation of claim 12, which further comprises a pharmaceutically acceptable carrier.

14. A method for introducing a nucleic acid into a cell comprising the step of contacting a cell with a composition of anyone of claims 1 to 11 or with a formulation of claims 12 or 13 whereby said nucleic acid (i) is taken up into said cell.

15. A method for treatment of a mammalian condition or disease comprising the steps of:
(a) administering to a mammal suffering from said condition or disease a therapeutically effective amount of a composition of anyone of claims 1 to 11 or with a formulation of claims 12 or 13 wherein said nucleic acid (i) is specific for the treatment of said disease, and
(b) permitting said nucleic to be incorporated into at least one cell of said vertebrate whereby said disease is effectively treated.

16. Use of a compound as defined in (iii) in claim 1 for the transfer of a nucleic acid into a cell.

17. Use of a compound as defined in (iii) in claim 1 for the preparation of a composition for introducing a nucleic acid into a cell.

18. The composition of any one of claims 1 to 10 for use as an active pharmaceutical substance.

19. Use of the composition of anyone of claims 1 to 10 for the preparation of a pharmaceutical formulation for the introduction of a nucleic acid into cells.
